(19) United States
(12) Patent Application Publication
(10) Pub. No.: US 2009/0163762 A1
(43) Pub. Date: Jun. 25, 2009

(54) DEVICE FOR THE REGENERATION AND PREVENTION OF DEGENERATION OF THE CARTILAGINOUS TISSUE AND SUBCHRONDRAL BONE, THE PROLIFERATION OF CHONDROCYTES AND FOR BLOCKING OR REDUCING THE FIBROBLASTIC EVOLUTION OF CHONDROCYTES AND MESENCHYMAL CELLS BY MEANS OF A PULSED ELECTROMAGNETIC FIELD

(76) Inventors: Stefania SETTI, Carpi (IT); Ruggero CADOSSI, Carpi (IT); Donata MARAZZI, Carpi (IT)

Correspondence Address: OSTROLENK FABER GERB & SOFFEN, 1180 AVENUE OF THE AMERICAS, NEW YORK, NY, 100368403, US

(21) Appl. No.: 12/274,757

(22) Filed: Nov. 20, 2008

Related U.S. Application Data
(63) Continuation-in-part of application No. 12/300,233.

(30) Foreign Application Priority Data
May 12, 2006 (IT) TO2006A000344

(51) Int. Cl. A61N 2/04

(52) U.S. Cl. 600/14; 600/9

(57) ABSTRACT

Device for the regeneration and prevention of degeneration of the cartilage and subchondral bone, the proliferation of chondrocytes and for blocking or reducing the fibroblastic evolution of chondrocytes and mesenchymal cells by means of electromagnetic waves comprising a device for generating a periodic signal u(t) and a power amplifier suitable for applying the signal u(t) to a pair of solenoids for the generation of an electromagnetic field M(t) addressed towards a portion of human/animal body containing cartilage. Setting means are provided for the generation of an electromagnetic field having intensity between 0.5 and 2 milliTesla, frequency between 37 and 75 Hz and period of application between 1 hour and 9 hours.

This is a continuation-in-part application of U.S. patent application Ser. No. 12/300,233, filed Nov. 10, 2008, by Stefania Setti and Ruggero Cadossi, entitled DEVICE FOR THE REGENERATION AND PREVENTION OF DEGENERATION OF THE CARTILAGINOUS TISSUE AND SUBCHRONDRAL BONE AND THE PROLIFERATION OF CHONDROCYTES BY MEANS OF A PULSED ELECTROMAGNETIC FIELD, the content of which is incorporated herein by reference.

### TECHNICAL FIELD

The present invention relates to a device for the regeneration and prevention of degeneration of the cartilaginous tissue and subchrondral bone and the proliferation of chondrocytes by means of a pulsed electromagnetic field. Further the present invention refers to a method for blocking, preventing or reducing the fibroblastic evolution and the expression of its chondrocyte phenotype and its mesenchymal cells used to repair the cartilaginous tissue by means of the application of a pulsed electromagnetic field. Lastly this invention concerns an electromagnetic field stimulator device for Anatomic Biophysical Chondroprotection.

### BACKGROUND ART

Techniques are known for therapeutic treatment of the human body by means of pulsed electromagnetic fields in which a solenoid is powered by a time-variable electrical signal (for example a current-variable signal) for the generation of a pulsed electromagnetic field which is addressed towards a portion of human body containing cartilaginous tissue/subchondral bone in which induced microcurrents form which contribute to the healing and/or improvement of inflammatory processes and/or lesions present in the portion of human body treated.

Electromagnetic field stimulator devices, in which a generator of pulsating, variable current is able to feed at least one solenoid for generating an electromagnetic field directed onto a portion of the human body comprising bone tissue, are well known.

For example, American patent U.S. Pat. No. 3,820,534 published in 1974 describes a device able to allow the growth and repair of bone tissue via the electromagnetic field generated by a solenoid powered by an alternating electric signal with a frequency of less than 50 Hz.

Scientific observations have been reported which suggest the possibility of useful application of pulsed electromagnetic fields for the treatment of articular cartilage and subchdondral bone.

Said techniques have not succeeded, however, in producing devices that can be used for a real therapeutic cycle of the cartilaginous tissues/subchondral bone for application in humans, i.e. able to act and modify in a sensitive, specific and significant way the structure of the damaged or inflamed cartilage and subchondral bone.

The lack of adequate preclinical experimentation permitting identification and combination of the optimal, i.e. effective, electromagnetic field parameters (amplitude, waveform frequency and duration of exposure) can result in incorrect choice of the parameters that characterise the electromagnetic field, therefore not obtaining any therapeutic effect, as also highlighted in literature (Fini M et al, Effects of pulsed electromagnetic fields on articular hyaline cartilage: review of experimental and clinical studies. Biomed Pharmacother.2005 August; 59(7):388-94. Review), or where often the only effect observed is related to a reduction in the pain symptomotology, without any attempt to modify the trophism of the articular cartilage. (Thansborg G et al., Treatment of knee osteoarthritis with pulsed electromagnetic fields: a randomized, double-blind, placebo-controlled study. Osteoarthritis and Cartilage. 2005 July; 13(7):575-81. Peroz I et al, A multicenter clinical trial on the use of pulsed electromagnetic fields in the treatment of temporomandibular disorders. J Prosthet Dent. 2004 February; 91(2):180-7.).

The articular cartilagenous tissue is a tissue characterized by a great resistance and elasticity, that plays a structural support role within the organism as well as the role of absorbing loads applied to an articulation. It is a connective tissue made of cells dispersed in an abundant gel-like extracellular matrix, rich in fibres, hyaluronic acid, responsible for the elasticity, and of an amorphous substance of proteic and collagene origin. The synthesis of the substances that make up the matrix and of the matrix structure itself is carried out by the cells it consists of, the chondrocytes.

Specifically the chondrocytes are able to secrete a complex mixture of collagen fibres and proteoglycans that is structured in a reticular form giving origin to the intercellular matrix, determining the solid but elastic consistency, characteristic to this tissue.

The first manifestation of the differentiation in the cartilage is the appearance of mesenchymal cell clusters that differentiate into chondroblasts. The chondroblasts then become chondrocytes.

As compared with other connective tissues, the cartilage is a non vascularized tissue; chondrocytes are fed by diffusion through the extracellular matrix and therefore the cartilage is a tissue that grows and regenerates itself much more slowly than other tissues, in particular it shows limited capacity to repair a cartilagenous damage.

As is known, the degeneration of articular cartilage manifests itself very frequently and it progressively worsens with age. Suffice it to think that alterations of the cartilage surface only manifest themselves in 5% of the population below 25 years of age, while they are present in more than 80% of people over 75. However, cartilage degeneration is not just a consequence of ageing, but also the end result of a complex series factors related to problems of a biological nature and/or mechanical problems.

Articular cartilage does not possess significant self-healing capabilities other than for small lesions when in youth. The quality and mechanical properties of articular cartilage can only diminish in the course of life.

Of the causes that can damage articular cartilage, we can identify those with a mechanical basis and those with a biological basis

Mechanical causes can be acute or chronic, depending respectively on whether the result of a severe trauma or an alteration of the load axis.

The biological causes are mainly due to the presence of inflammation ascribable to subchondral bone and intraarticular structures (synovial in particular). Inflammatory processes produce a strong catabolic effect on cartilage, because the inflammatory cells synthesize and release pro-inflammatory cytokines (interleukin 1 and 6, and TNF-□) that inhibit the synthesis of proteoglycans by the chondrocytes and increase the synthesis of enzymes (matrix metalloproteinase 3, MMP3) which in turn degrade the cartilaginous matrix. The inflammatory response of the articular structures is often the consequence of acute or chronic traumas, distortions, avascular bone necrosis of the subchondral bone, bone marrow edema of the condoles, and side effects of open or arthroscopic surgery.

Post-traumatic or degenerative damages to articular cartilage represent an extremely common pathology, that involves a great number of subjects and is the initial phase of the arthrosic degenerative pathology.

The techniques mostly used for the treatment of cartilagenous lesions are of a surgical nature, such as for example cartilage cleansing in arthroscopy or damaged cartilage tissue abrasion, the so called chondroplasty.

These techniques, although they give good results, only allow to achieve the formation of a new fibro-cartilagenous tissue with morphological and structural properties which are quite different, worse than those that identify the cartilage of the articular surface achieving only a slowing down of the arthrosic process.

Better results are achieved through the transplant of autologous chondrocytes which at present represents the best solution, especially for treating severe cartilage defects.

The use of staminal mesenchymal cells contained inside the midollar stroma, the connective or adipose tissue which are collected and laboratory expanded in order to achieve a clinically relevant number thereof, belongs to this type of intervention of regenerative medicine. Outside the body (ex vivo) the mesenchymal staminal cells maintain a good proliferative capacity and are able to adhere to glass or plastic surfaces, which are commonly used for the culture of cells in labs.

In order to obtain an expansion, the cells are cultivated and treated with growth factors obtaining the differentiation of the mesenchymal cells into chondrocytes.

It has however been observed how the differentiation of the cells and of the phenotypic expression is influenced not only by growth factors but also by the microenvironment they are placed in, especially in vivo. When the cells are implanted in vivo in an inflammatory microenvironment, characterized by the presence of molecules with pro-inflamatory activity, it has been observed the transplanted cells, chondrocytes and mesenchymal cells, evolve into a fibroblastic phenotype that determines quite a great reduction of the elastic properties of the tissue. The presence in the microenvironment of pro-inflamatory stimula favours also the synthesis and the release of molecules such as the prostaglandins that increase apoptosis (programmed death) of the chondrocytes.

On the basis of these premises, it becomes fundamental to have a therapy at hand capable of locally controlling the inflammation, at both the subchondral bone and articular structure levels. The therapy must also be able to act directly on the chondrocytes in the depth of the cartilage to prevent the catabolic effects of the inflammatory cytokines on the chondrocyte and on the matrix, to facilitate anabolic activities and the synthesis of proteoglycans. The simultaneous treatment of cartilage, subchondral bone tissue and articular fleshy structures is only practicable with physical means.

### DISCLOSURE OF INVENTION

The object of the present invention is to provide a device capable of generating a pulsed electromagnetic field, the parameters of which are chosen and optimised on the basis of preclinical studies which evaluate the regeneration and prevention of degeneration of the cartilaginous tissue and subchondral bone; said preclinical studies also evaluate the proliferation of chondrocytes, in order for the device to be usable in a therapeutic type process which obtains tangible results that are clinically relevant and applicable to humans.

The device can also be used in conjunction (before or after) with the administration of pharmacological agents (drugs, growth factors) aimed at stimulating cartilaginous repair, or also in conjunction with treatment of the subchondral bone by means of microfractures.

A further object of the present invention is to provide a method that allows the control of the differentiation of the chondrocytes and of the mesenchymal cells avoiding their fibroblastic differentiation and evolution.

The preceding object is achieved by the present invention since it relates to a use of a device comprising means for generating a periodic signal/periodic signals u(t) and power amplifying means adapted to apply said signal u(t) to at least one solenoid for the generation of a pulsed electromagnetic field M(t) addressed towards a portion of human/animal tissue containing cartilage, to block or reduce the fibroblastic evolution of chondrocytes and mesenchymal cells.

According to a second aspect of the present invention, it is provided a method for blocking or reducing the fibroblastic evolution of chondrocytes and mesenchymal cells by means of electromagnetic waves comprising the steps of generating a periodic signal/periodic signals u(t) and applying said signal u(t) to at least one solenoid for the generation of a pulsed electromagnetic field M(t) addressed towards a portion of human/animal tissue containing cartilage.

The device according to the invention can be used alone or in association with therapeutic agents, preferably selected from the group consisting of growth factors, cytochines, which are the basic constituents of the matrix.

Advantageously the use according to the present invention allows to prevent or reduce the differentiation into the fibroblastic phenotype, blocking inflammatory phenomena and thus promoting recovery from a cartilagenous lesion that produces a functional reparative tissue consisting in hyaline cartilage following the activity of the cells that contain a chondrocytal phenotype. The present invention allows moreover to preserve the cartilagenous tissue which is not directly compromised by the lesion, which being exposed to the negative effects of the inflamatory environment could undergo degeneration. Moreover the inhibition of the prostaglandin synthesis according to the present invention protects against and prevents chondrocytes apoptosis. The device moreover allows to increase the proliferation and then the expansion of the number of transplanted cells, chondrocytes and mesenchymal staminal cells, making them more sensible to the activity of growth factors.

### BRIEF DESCRIPTION OF THE DRAWINGS

The invention will now be illustrated with particular reference to the accompanying drawings which represent a preferred non-limiting embodiment in which:

FIG. 1 illustrates a simplified wiring diagram of a device for the regeneration and prevention of degeneration of the cartilaginous tissue and subchondral bone, the proliferation of chondrocytes and for blocking or reducing the fibroblastic evolution of chondrocytes and mesenchymal cells by means of a pulsed electromagnetic field produced according to the precepts of the present invention;

FIGS. 2, 3 and 4 illustrate parameters that can be controlled by the device of the present invention;

FIGS. 5a, 5b, 6a, 6b and 12 illustrate results obtained with the device of the present invention;

FIGS. 7 and 8 illustrate statistical analyses performed on the data obtained with the device of the present invention;

FIGS. 9, 10 and 11 show support means to make the device of FIG. 1 portable by a person;

FIG. 13 illustrates a simplified wiring diagram of an electromagnetic field stimulator device for Anatomic Biophysical Chondroprotection realized according to the principles of this invention;

FIG. 14 illustrates the time modulation of two quantities controlled by the stimulator device in accordance with this invention,

FIG. 15 illustrates intracellular events activated by the stimulator device in accordance with this invention,

FIGS. 16 and 17 show histograms regarding the differences that are found at intracellular level between regions treated with the stimulator device in accordance with this invention and regions not subjected to treatment,

FIGS. 18a and 18b respectively show an image of an untreated articular region and an image of an articular region treated with the stimulator device in accordance with the invention, and

FIGS. 19a and 19b show images of an osteo-cartilaginous graft treated with the stimulator device in accordance with the invention.

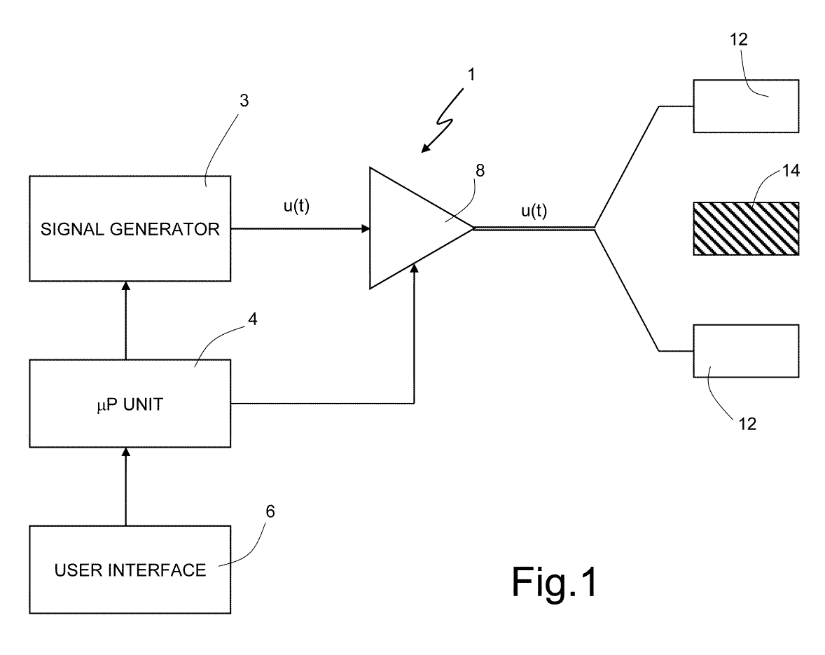

With reference to FIG. 1, 1 indicates, as a whole, a device for the regeneration and prevention of degeneration of the cartilaginous tissue and subchondral bone, the proliferation of chondrocytes and for blocking or reducing the fibroblastic evolution of chondrocytes and mesenchymal cells by means of a pulsed electromagnetic field.

The device 1 comprises a signal generator device 3 (of known type) controlled by a microprocessor unit 4 and suitable for outputting a periodic type signal u(t).

The microprocessor unit 4 is connected to a user interface 6 (for example keyboard/video) for selection of the waveform (square wave, saw tooth, linear ramp, etc.) of the signal u(t) and adjustment of the frequency and duty-cycle of said signal u(t). The user interface 6 also permits generation of the signal u(t) for a time interval Tmax that can be selected as required.

The signal generator device 3 is connected at the output to the input of a variable gain power amplifier 8 which outputs a power signal U(t) transmitted to a pair of solenoids or to one single solenoid (of known type) 12, appropriately curved and modelled. In some embodiments the solenoid 12 can be one single solenoid.

During use, a portion of human/animal body containing a portion of cartilage/subchondral bone 14 to be treated is positioned between the pair of solenoids 12. A culture (not illustrated) of chondrocytes can also be positioned between the solenoids 12.

The power amplifier 8 is controlled by the microprocessor unit 4 so that via the user interface 6 it is possible to adjust the amplitude of the signal U(t) transmitted to the solenoids and therefore the intensity of the electromagnetic field M(t) acting on the cartilage 14.

Preferably but not exclusively each solenoid 12 consists of a support made of a sheet of flexible material on which a trace of conductive material (for example copper) is deposited, forming the coils of the solenoid. Alternatively, the solenoid could be made with a reduced number of coils (for example below 200) so that it can be modelled for good adhesion to the articular surface.

According to the present invention it is possible, via the user interface and the microprocessor unit 4, to set the device 1 so that it generates a pulsed electromagnetic field with peak intensity between 0.5 and 2 milliTesla.

The medical studies carried out by the applicant have highlighted that the identification of said limited power interval (0.5 and 2 milliTesla) permits generation of an electromagnetic field M(t) which results in effective regeneration and/or prevention of degeneration of the cartilage and subchondral bone.

In further detail, the electromagnetic field can have a power of between 1 and 2 milliTesla.

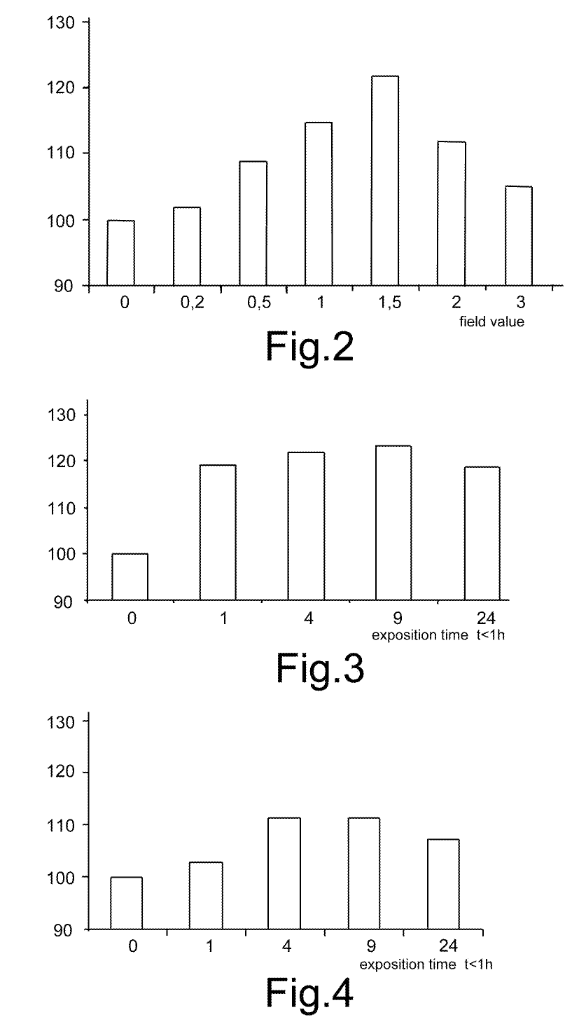

In particular the electromagnetic field can have a power of around 1.5 milliTesla, as highlighted in FIG. 2 which shows on the X axis the value of the electromagnetic field and on the Y axis the synthesis of proteoglycans which indicate an anabolic effect on the cartilage and in particular on the cartilaginous matrix; the value of 1.5 m-Tesla permits maximization of regeneration and prevention of degeneration of the cartilage and subchondral bone.

As is known, a high synthesis of proteoglycans is an indicator of synthesis activity of the extracellular matrix of the articular cartilage.

The studies of the applicant have shown that correct adjustment of the field intensity is the main factor for obtaining a correct process of regeneration and prevention of degeneration of the cartilage and subchondral bone.

The studies of the applicant have also shown that, subordinately to the intensity, also the frequency of the signal applied constitutes a factor for control of the processes of regeneration and prevention of degeneration of the cartilage and subchondral bone.

Indeed, according to a further embodiment of the present invention it is possible, via the user interface and the microprocessor unit 4, to set the device 1 so that it generates a signal u(t) having variable frequency below 100 Hz.

The studies carried out by the applicant show that a signal with frequency above 100 Hz results in an inefficient process of regeneration and prevention of degeneration of the cartilage and subchondral bone.

Preferably, the frequency of the signal u(t) is set so as to present a frequency of between 2 Hz and 75 Hz.

In further detail, the frequency can be between 37 Hz and 75 Hz, the frequency interval in which the greatest therapeutic effect is produced.

The interval 37-75 Hz permits maximization of the processes of regeneration and prevention of degeneration of the cartilage and subchondral bone as highlighted in FIG. 3 which shows on the X axis the frequency value of the signal u(t) and on the Y axis the synthesis of the proteoglycans.

Lastly, the studies of the applicant have highlighted that, subordinately to the intensity and frequency, the duration of the treatment also constitutes a factor for control of the processes of regeneration and prevention of degeneration of the cartilage and subchondral bone.

In particular, via the user interface and the microprocessor unit 4, the device 1 is set so that it generates an electromagnetic field for a variable time interval, if possible less than 9 hours. Preferably the setting interval of the electromagnetic field is between 4 and 9 hours as highlighted in FIG. 4 which shows on the X axis the application time (expressed in hours) and on the Y axis the synthesis of proteoglycans.

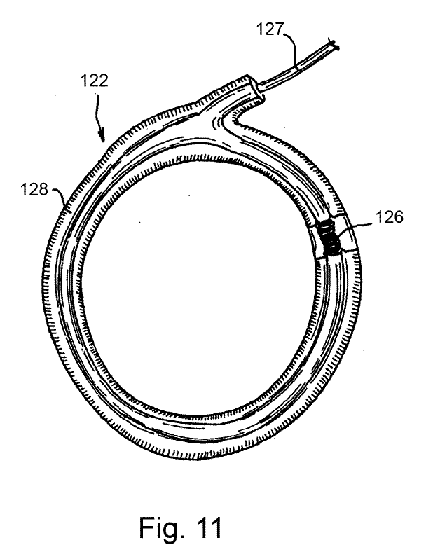

In FIG. 13, an electromagnetic field stimulator device for Anatomic Biophysical Chondroprotection (CBA) is generally indicated by 201.

In particular, the stimulator device 201 includes a synchronizing signal generator 203 suitable for producing an output signal ck with a constant frequency, for example 16 MHz, used as an internal reference. The device 201 also includes a time-division circuit 204 receiving the synchronization signal ck in input, and able to time divide the signal ck to generate a scanning signal sc fed in input to a table 207.

The table 207 contains a number of selectable maps, each of which implements a function f(t) that provides, for each value of the scanning signal sc in input, an output value Iout that expresses a target current intensity.

In greater detail, the function f(t) is linear and represents a ramp with a certain slope that provides, for increasing values of the scanning signal sc in input, linearly increasing intensity values for the target current Iout. At the end of scanning the function, the function is scanned again starting from the beginning of the ramp. In this way, following operation of the synchronizing signal generator 203 and the time divisor 204, the output signal Iout presents a saw-tooth profile comprising the repetition of a ramp that expresses increasing values of current intensity.

The device 201 also includes an attenuator circuit 2010 that receives the signal Iout in input and feeds it to a subtraction block 2012, which performs the arithmetic difference between the same signal Iout and a signal Imis that expresses the real current intensity. The output of the subtraction block 2012 (ring error) is fed to the input of an error amplifier 2014 (for example, a Proportional-Integral-Derivative circuit) which has an output that pilots a pulse width modulator block (PWM) 2018 via a digital/analogue converter 2019.

The PWM block 2018 generates an alternating analogue signal s(t) in output with a constant frequency (for example, 250 KHZ) and adjustable duty cycle. For example, the signal s(t) could have a square waveform.

The duty cycle of the signal s(t) is modified as a function of the numeric value fed in input to the PWM block 2018; in particular, the duty cycle of the signal s(t) increases with the increase in the numerical value of the signal fed in input to the PWM block 2018.

The analogue signal s(t) is fed in input to an output amplifier stage 2020 (of known type), which generates an output power signal S(t) that feeds a solenoid 2024 via a low-pass filter 2022. The low-pass filter 2022 is suitable for eliminating spurious components from the power signal S(t); this filter 2022 is advantageous as the power signal S(t) generates high-order harmonic components.

The solenoid 2024 generates a special type of electromagnetic field (detailed further on) that is directed onto a portion of a human body 2026 comprising a portion of cartilage 2027, especially articular cartilage.

The solenoid 2024 is realized in a manner such that the physical stimulus can follow the shape of the anatomic surfaces of the portion of the human body 2026 and can penetrate in depth into the cartilage and subchondral bone.

In particular, the solenoid 2024 can be opportunely made using multiple sheets of a flexible material (for example, three sheets of Kapton, 50 micron thick), on the faces of which copper tracks have been deposited, via a photoengraving process, which form the turns of the solenoid 2024 itself. For example, the copper tracks can be conveniently distanced 0.3 mm from each other, be 1.7 mm wide and 35 μm thick.

The distance between the copper tracks and their thickness and width render the solenoid 2024 particularly flexible, thereby permitting the physical stimulus to be transmitted over the entire zone to be treated in a uniform manner all around the zone of application, following the shape of the anatomic surfaces.

In particular, the electromagnetic field induced by the solenoid 2024 distributes itself over the portion of the human body 2026 in such a way to include not just the cartilaginous tissue in all of its extension and all of its thickness, but also the various articular surfaces, meniscuses, ligaments, symposia, subchondral bone, etc.

A detector device 2028 (for example, a shunt resistor or a Hall-effect sensor) detects the value of the current i(t) running in the solenoid 2024. The output of the detector device 2028 feeds a feedback amplifier 2029, the output of which, in turn, feeds an analogue/digital converter 2030, which produces the signal Imis that expresses the measured value of the current running in the solenoid 2024.

The attenuator circuit 2010 proportionally reduces all of the points in table 207 by a programmable parameter IPK to achieve a uniformly scaled current profile. In particular, if the value of the parameter IPK is equal to zero, no limitation on the current feeding the solenoid 2024 is applied, which is thus free of restrictions, i.e. it is the maximum load request. However, if the parameter IPK is non-zero, this parameter IPK represents instead the maximum peak value for the current generated by the solenoid 2024. Each value in the table 207 will therefore contribute to realizing a current value proportional to the maximum peak value expressed by the parameter IPK.

In use, after the device 201 is switched on, a signal Iout is generated that has a reference function and comprises the repetition of a ramp that represents increasing values of current intensity. The reference value Iout can also be altered by selecting a different map in table 207.

The PWM block 2018 receives a variable signal in input and consequently changes the duty cycle of the power signal S(t) in function of this input signal, in order to induce a current in the solenoid 2024 that follows the modulation established by the signal Iout, which thus performs a reference function.

The intensity of the current in the solenoid 2024 is therefore regulated via the variation of the duty cycle of the power signal S(t).

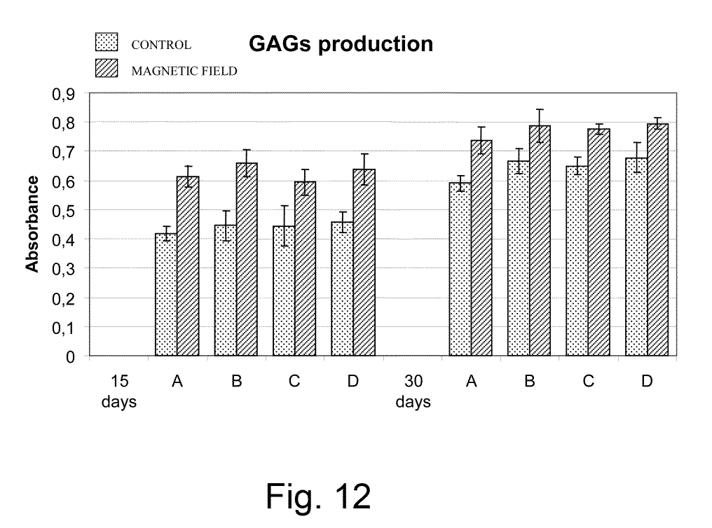

In this way, a current generator is realized that feeds the solenoid 2024 with a current i(t) whose waveform includes the repetition of a ramp (FIG. 14) having a predetermined and constant slope.

This current causes the generation of an electromagnetic field that induces on a control probe 2032 (FIG. 13) irradiated by this electromagnetic field, an induced voltage Vin of markedly constant amplitude during the ramp-like linear growth period of the current in the solenoid 2024.

The induced voltage is in fact proportional over time to the derivative of the signal feeding the solenoid 2024.

For example, an induced voltage Vin having constant amplitude between 1 and 4 Millivolt during the entire active period of piloting the solenoid 2024 can be usefully realized.

The feedback system of the device 201, constituted by the detector device 2028, the feedback amplifier 2029 and the analogue/digital converter 2030, accomplishes continuous monitoring of the current i(t) circulating in the solenoid 2024 and compares (subtraction block 2012) the measured current value Imis with that “mapped” in the table 207, i.e. with the signal Iout.

In the case of variances from these values, due to small variations in impedance (resistance and/or inductance) of the solenoid 2024 for example, the feedback system immediately takes care of, via the ring error signal, the correction to the duty cycle of the power signal S(t) and thus the value of the current feeding the solenoid 2024, in order to maintain the waveform of the induced voltage Vin unaltered.

Experimental results of the applicant have shown that the device 201 achieves Anatomic Biophysical Chondroprotection, or rather that it is capable of: preserving the integrity of cartilage, controlling inflammatory articular processes dependent on both subchondral bone and articular structures, protecting the chondrocyte and the cartilaginous matrix from the catabolic effects of inflammatory cytokines, favouring cartilage trophism stimulating the chondrocytic metabolism and the synthesis of proteoglycans, and acting directly on subchondral bone protecting trophism and guaranteeing integration in the presence an autologous transplant.

In particular, Anatomic Biophysical Chondroprotection finds favourable application in human beings for the treatment of inflammatory and degenerative conditions regarding articular cartilage and subchondral bone of the main articulations, especially the knee, in all conditions of bone marrow edema regarding the subchondral bone of femoral condoles, in the healing and integration of bone grafts after ligament reconstruction operations on the fibrous flexor sheaths of the knee, and in the healing and integration of knee joint osteo-cartilaginous grafts.

These effects are confirmed by a series of studies carried out both in vitro and in vivo, the results of which are detailed below.

Further aspects and advantages of the present invention will be apparent from the following description of several practical embodiments thereof given by way of non-limiting examples.

### EXAMPLES

### Example 1

### Method for the Treatment and/or Prevention of Pathologies Affecting the Cartilage and/or Subchondral Bone and for the Proliferation of Chondrocytes

The method was tested in vitro on bovine cartilage which has a high affinity with human cartilage.

Explants of articular cartilage in the form of discs were performed from five different animals aged between 14 and 18 months.

In particular, four explants were performed on each donor animal taken from areas near the same joint thus obtaining twenty discs.

Each group of explants was divided at random into a first subgroup of explants with test function (therefore subject to the electromagnetic field) and a second group of explants with control function (therefore not subject to the electromagnetic field).

The explants underwent pre-treatment by placing them for 48 hours in a culture of DMEM/F12 to which 10% of FBS (Fetal Bovine Serum) and antibiotics (penicillin 100 units/ml, streptomycin 0.1 mg/ml) (Life Technologies Paisley, U.K.) were added.

Subsequently the explants were placed for an additional period of 48 hours in a medium without serum at 37° C. in an atmosphere containing 5% of CO₂.

During the treatment each cartilage disc was placed between the solenoids 12 so that the plane of the solenoids was perpendicular to the discs and the direction of the electric field induced in the disc was perpendicular to the electromagnetic field.

The device 1 was used adjusting the intensity of the electromagnetic field, the frequency of the signal u(t) and the application time as illustrated above. The intensity of the electromagnetic field produced was measured with a Hall effect sensor of a gaussmeter.

In said regard, at the end of the period of equilibrium in culture illustrated above, the explants were exposed for: 1, 4, 9, 24 hours to a pulsed electromagnetic field obtained with the device 1.

Exposure to the pulsed electromagnetic field was performed with 10% FBS in the culture medium (0.5 ml/well). The evaluations were performed after 24 hours, independently of the exposure period.

The cultures not exposed to pulsed electromagnetic field (control cultures) were arranged in the same incubator as the one containing the cultures subject to electromagnetic field.

Synthesis of the proteoglycans was measured by incorporating a radioactive sulphate into the glycosaminoglycans (GAGs) which, as is known, are basic biochemical components of the proteoglycans.

The radioactive compound 5 μCi/ml of Na₂—³⁵SO₄ (2.2 mCi/ml) (produced by the company Amersham Pharmacia Biotech, Buckinghamshire, England) was added at time 0 both to the explants subject to treatment by pulsed electromagnetic field and to the control explants not subject to pulsed electromagnetic field, thus performing radio-marking.

After the radio-marking, the explants were washed and digested in a buffer containing 20 mM of phosphate (pH 6.8) and 4 mg/ml of papain (produced by the company Sigma-Aldrich S.r.l. Milan, Italy) and kept at 60° C. for 12 hours.

The content of the proteoglycans marked with compounds of radioactive sulphur ³⁵S belonging to new synthesis proteoglycans PGs (³⁵S-PGs) was measured following precipitation of the radioactive sulphur compound ³⁵S-PGs by means of cetylpyridinium chloride (said compound is available from Sigma-Aldrich S.r.l. Milan, Italy) and filtering on fibreglass (Whatman GF/C).

The filters were then dried and the radioactive sulphur compounds were quantified by scintillator count. The quantity of proteoglycans synthesised as a result of the cellular activity or activity of the chondrocytes was thus identified.

On the basis of the results of the experiments an exposure time of between 1 hour and 9 hours was identified. In further detail, the maximum therapeutic effect is obtained with an exposure field of between 4 and 6 hours.

Subsequently synthesis of the proteoglycans was measured in the explants of cartilage using pulsed magnetic fields having different peak values of between 0.2 mT and 3 mT.

This permitted selection of the interval between 0.5 and 2 mTesla which defines a first therapeutic treatment window. The results of the tests also permitted definition of the sub-interval between 1 and 2 mT (second treatment window) and the peak value of 1.5 mT which maximises the effects of the treatment.

Lastly, following selection of the best exposure time and preferred electromagnetic field value, synthesis of the proteoglycans was measured with different frequencies (0, 1, 2, 37, 75, 110, 150, 200 Hz). This enabled us to ascertain that for frequencies above 100 Hz no appreciable therapeutic effects are obtained.

The sub-interval between 2 and 75 Hz and the sub-interval between 37 and 75 Hz in which the therapeutic effect is maximised were then selected.

On the basis of the results obtained by means of a first set of experiments, the explants were exposed for 9 hours to a pulsed electromagnetic field, the amplitude of which was around 1.5 milliTesla.

For said pulsed field value an unexpected synthesis of proteoglycans was found (approx. 50% more in the implants subject to pulsed electromagnetic field compared to the findings for the implants not subject to electromagnetic field).

Once the most effective window for each parameter of the pulsed electromagnetic field had been identified, the investigations were extended.

The studies performed by the applicant on sheep which underwent osteochondral transplant of the knee also showed that the action of the device 1 according to the present invention determines a rapid recovery of the subchondral bone tissue and prevents bone re-absorption phenomena, creating optimal conditions for viability and survival of the overlying articular cartilage.

Good integration of the transplanted bone tissue prevents the formation of small bone cysts thus guaranteeing stability of the bone graft in the long term. In this regard it should be noted that, in the case of osteocartilaginous transplants, early fixing of the subchondral bone is the necessary condition for viability and preservation of the cartilage transplanted and success of the operation.

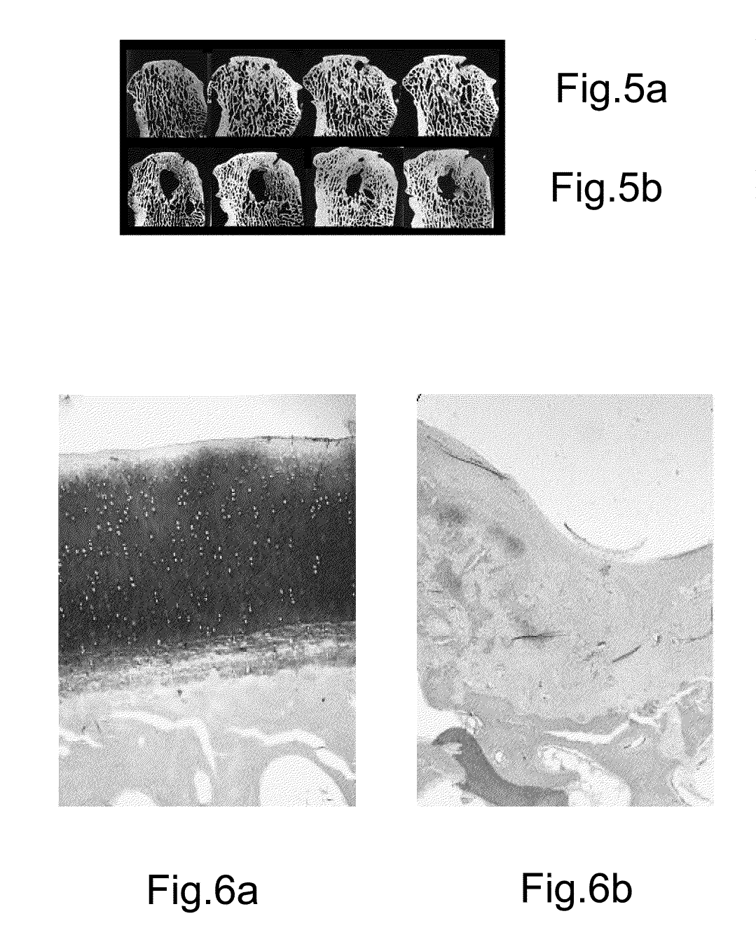

FIGS. 5a, 5b illustrate radiographic images of an osteocartilaginous graft.

In particular, FIG. 5a refer to an osteocartilaginous graft stimulated with device 1: in said figures the optimal integration of the transplant throughout the thickness as shown by the different sections can be observed.

FIG. 5b refer to an osteocartilaginous graft not stimulated with device 1: in said figures areas of re-absorption in the different sections can be observed.

In particular in the microradiographic image of FIG. 5a complete integration of the subchondral bone can be noted.

The percentage of bone re-absorption areas (dark) in the transplanted cylinders of the stimulated animals is 31%, against 60% bone re-absorption areas in the transplanted cylinders of the control animals.

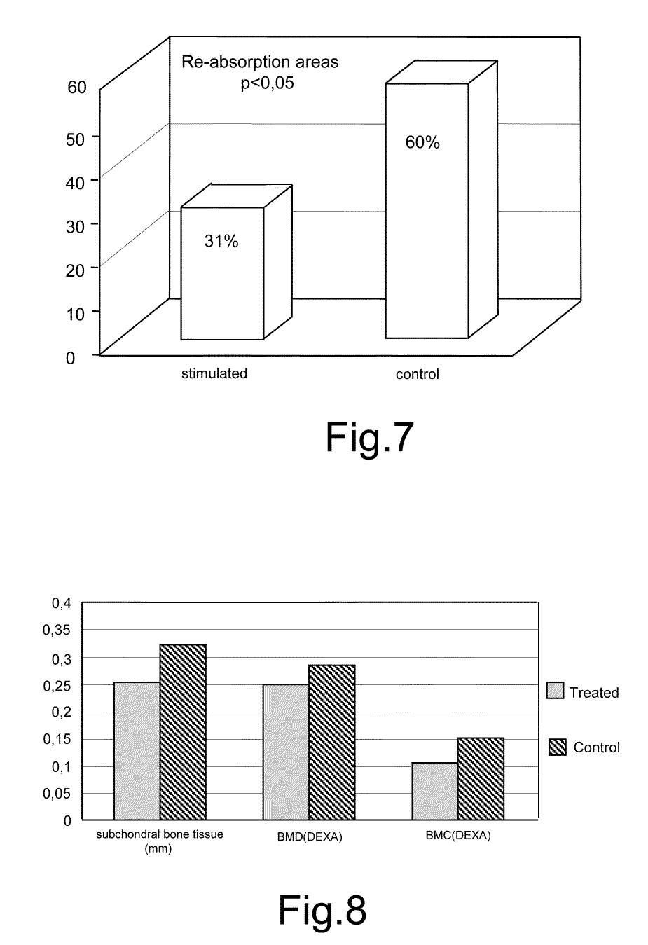

The histogram of FIG. 7 illustrates the percentage of bone re-absorption areas present in the transplanted cylinders of the stimulated and control animals.

This figure shows that the pulsed electromagnetic fields are able to promote early fixing of the graft, guaranteeing optimal integration of the transplanted bone tissue, preventing the formation of small bone cysts, hence ensuring stability of the bone graft and therefore success of the operation.

The histological images 6a, 6b furthermore illustrate a section of the transplanted cartilage (FIG. 6a) treated with the device of FIG. 1 in which the viability of the transplanted cartilage, which has an adequate thickness and intense colouring of the cartilaginous matrix, is evident.

In particular FIG. 6a highlights the presence of hyaline tissue, while FIG. 6b (non-treated cartilage) highlights the presence of fibrous, fibrocartilaginous tissue.

In the transplants treated with the device 1 the formation of fibrous tissue is clearly inferior with respect to the non-treated controls: 15% as against 32%.

Lastly, the applicant has demonstrated that treatment with the device 1 can effectively prevent cartilaginous degeneration in experimental animals (Dunkin Hartley), maintaining functionality. Animals with initial osteoarthrosis, aged 15 months, were treated for 6 months. Not only did treatment with the device 1 prevent degeneration of the cartilage, it also prevented osteoschlerosis of the subchondral bone, indicating that the cartilage had maintained its mechanical characteristics. Indeed, when the cartilage loses the ability to absorb stress due to the load, the stress is transmitted directly to the subchondral bone tissue which reacts by increasing its density and thickness. The table shows that the histological evaluation (Mankin score) of the cartilage treated with the device 1 is clearly inferior (therefore better) than in the control animals.

| | | Animals treated with |
|---|---|---|
| | Control animals | electromagnetic fields |
| Mankin score | 13.8 ± 1.1 | 4.6 ± 1.5*** |

The histomorphometric and bone density measurements by means of Dual Energy X-ray Absorptiometry (DEXA) highlight a lesser density and sclerosis of the subchondral bone tissue in the animals treated with the device 1 (FIG. 8).

Lastly, experiments carried out by the applicant have highlighted that the electromagnetic field generated by the device 1 according to the above procedures is effective in stimulating the proliferation of chondrocytes cultivated in vitro.

Said chondrocytes can be used in different techniques, for example they can be used to perform Autologous Chondrocyte Implantation (ACI), a method introduced in the eighties by Petterson to promote healing of the cartilage.

Said method provides for an initial collection of autologous chondrocytes by means of arthroscopy from patients affected by chondral lesions. The chondrocytes are then isolated by digestion of the cartilaginous matrix and cultivated in vitro, after dedifferentiation towards the chondroblastic phenotype.

In the ACI technique, the cells thus obtained are then transplanted, in the form of suspension, into the patient's joint below a periostal flap sutured to the chondral cartilage during the operation.

Alternatively, the chondrocytes can be used in the MACI (Matrix-Induced Autologous Chondrocyte Implantation) technique which involves initial arthroscopy and in vitro cultivation of autologous chondrocytes: the chondroblasts thus obtained are scattered three weeks after collection on a type I and III pig collagen scaffold. This “membrane” can be implanted on the chondral lesion of the patient and affixed via the use of fibrin glue.

The applicant has been able to demonstrate that on the one hand treatment with the device 1 (and with the parameters highlighted above) stimulates the proliferation of these cells which are transplanted. Stimulation of proliferation represents a fundamental element for colonisation of the cartilaginous lesion site to be treated.

Lastly it is important to remember the role of the stem cells in the healing process of a cartilaginous lesion, as demonstrated by the techniques that involve making small perforations in the subchondral bone at the base of cartilaginous lesions. The aim is to promote the migration of totipotent cells, from the bone marrow to the surface of the subchondral bone, so that they can provide the necessary biological support for healing.

The applicant has carried out studies on stem cells obtained via the process briefly illustrated above, in order to highlight that the device is able to stimulate the proliferation, migration and ability thereof to colonise a substrate used in the treatment of cartilaginous lesions.

According to a preferred and independent aspect of the present invention, the device 1 is coupled with support means to make the device 1 portable by a person.

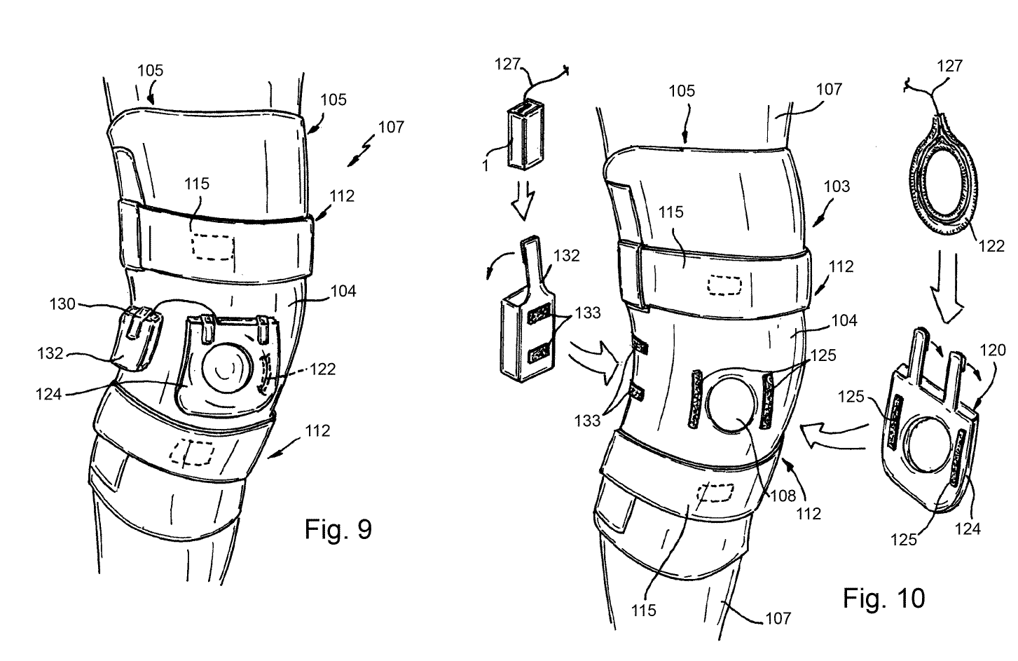

These supporting means comprise (FIGS. 9, 10 and 11) a supporting body 103 defined by at least one contoured wall 104 defining a cavity 105 for housing a portion of the human body.

In the non-limiting example shown, wall 104 is shaped to define a kneepad, which defines the elongated cavity 105 for housing a leg portion 107 of a patient (not shown in full) close to the knee 108. Cavity 105, however, may obviously house any portion of the human body, e.g. an arm, shoulder, etc. Wall 104 is preferably made of flexible synthetic material to adapt to the contour of the human body, and is obviously also made of anti-allergic, nontoxic material, such as neoprene.

Supporting means also comprises an elastic connecting device 112 fitted to supporting body 103 to secure contoured wall 104 firmly to the portion of the human body. In the example shown, the elastic connecting device comprises two elastic straps 115, each having a portion fixed (e.g. stitched) to wall 104, and each having a fastening device, e.g. of VELCRO™, at the ends. Fastening devices other than those shown, however, may obviously be used.

Supporting means also comprises a seat 120 for housing a the solenoid 12 of device 1 located adjacent to contoured wall 104 and therefore close to the portion of the human body.

In the example shown, seat 120 is defined by a square pouch structure 124 made of fabric and connectable to an outer surface of contoured wall 4 by two reversible connecting devices 125, e.g. of VELCRO™, so that seat 120 is secured firmly in a predetermined position to contoured wall 104 when connecting devices 125 are connected firmly.

Solenoid 12 is conveniently made using a coiler (not shown) which forms a coil 126 (FIG. 11) comprising roughly 200 turns of copper wire with an average turn of 40 cm. Coil 126 is then wound with cotton tape to keep its shape. The two ends of coil 126 are connected to a bipolar power cable 127 of solenoid 122. Coil 126 is then covered with heat-sealed multilayer plastic material 128 comprising high-density sponge inside and a PVC sheet outside. Solenoid 122 is wound in air.

Solenoid 12 is conveniently powered by the device 1, which may also be housed in a pouch 132 fixed to the outer surface of wall 104 by a releasable connecting device 133, e.g. of VELCRO™.

In a variation not shown, more than one seat may be formed to house further solenoids. For example, two separate seats may be formed for two solenoids located, in use, on opposite sides, one medial and one lateral, of the joint for treatment, which is an advantageous arrangement for treating the knee joint. A single solenoid is mainly indicated for treatment of the kneecap, and two solenoids for treatment of larger areas of the joint or for patients with larger than average joints. Two solenoids therefore ensure a uniform induced signal over the whole joint, even in patients with more extensive lesions.

### Example 2

### Method for Blocking or Reducing the Fibroblastic Evolution of Chondrocytes and Mesenchymal Cells

A sample of bone marrow was collected from the acetabulum and femoral head of patients undergoing total hip arthroplasty. Bone marrow mononuclear cells were obtained by ficoll (Sigma, Italy) density gradient centrifugation and depleted of CD45+ and glycophorin-A (GlyA)+ cells using micromagnetic beads (Miltenyi Biotec, Italy). CD45−/GlyA− cells were plated in 75 cm2 culture flasks (Corning Inc, NY, USA) in mesencult+stimulatory supplement (both from StemCell, Technologies Inc, BC, Canada) and 1% penicillin-streptomycin (Gibco, Italy) for 14 days. Near-confluence cultures were then trypsinized and expanded through six sequential passages to confluence. At each passage, cells were characterised with the FACSCalibur flow cytometry system (Becton Dickinson, CA, USA) using antibodies against the following surface antigens: CD3, CD34, CD14, CD45, CD90 and CD105 (Becton Dickinson).

Cells were seeded onto the scaffold type I+II collagen matrices at an initial density of 1×10⁴/well and cultured in 12-well plastic plates (Nunc A/S, Denmark) for 30 days. Cells were grown in DMEM, 10% fetal bovine serum (FBS), 1% penicillin-streptomycin and 1% fungizone (all from Gibco), in 5% CO₂ atmosphere. The medium was replaced at 48 h intervals.

Cells were then exposed to a magnetic field generated by a pair of circular coils of copper wire placed opposite each other, in a perpendicular plane respect the plane of the plastic plates, which was placed between the two coils. Inside this area, the magnetic field is uniform and it is focused on the plastic plates. Taking into account the size of each plastic plates and its position with respect to the magnetic flux lines, the induced electric field inside each plastic plates is in a plane parallel to the multiwell plate surface. The coils are powered by the power amplifier 8, which produces the input voltage of pulse. The pulse duration of the signal is 1.1 ms and the repetition rate 75 Hz. The intensity peak of the induced electric field is detected with a standard coil probe (50 turns, 0.5 cm internal diameter of the coil probe, 0.2 mm copper wire diameter). The intensity of the magnetic field, 1.5 mT, is detected between two coils, by the Hall probe of the Gaussmeter (LE, Gaussmeter DG500, USA), with a reading sensitivity of 0.2%. The maximum variation of the electromagnetic field intensity, inside each well, is 1%. Control cultures (not exposed group) are placed inside the same incubator without any shielding, at a distance where no difference from background magnetic field is observed, when the PEMF generator is turned on.

Cell proliferation and glycosaminoglycans (GAGs) production were studied by spectrophotometric assay on days 15 and 30.

The studies carried out have demonstrated how the exposition of the cells to the electromagnetic field according to the present invention substantially increases the synthesis of the glycosaminoglycans, as shown in FIG. 12. This demonstrates that the mesenchymal staminal cells placed onto a type I or II collagene matrix differentiate into cells that express markers of the chondrocytes and when exposed to the electromagnetic field to a greater extent than control cells. The greater synthesis of GAG, that is of the chondrocytal activity is the result of the differentiation in a chondrocytal sense. The differentiation follows the proliferation.

### Example 3

### In Vitro Effects

### Inflammation Control

Anatomic Biophysical Chondroprotection acts in a specific manner on the adenosinic receptors A2A of the cellular membrane of pro-inflammatory cells, neutrophils, rendering then available to binding with adenosine. Within the sphere of adenosinic receptors, the receptors A2A are those of greater anti-inflammatory effect.

The bonding with adenosine causes: inhibition of the production of pro-inflammatory cytokines, reduction in the synthesis of free radicals, increase in the production of ATP and cytokines with anti-inflammatory action, TGF□, and the inhibition of cycloxygenase 2 activity.

The kinetic studies carried out by the applicant have shown how the stimulator device implemented in accordance with this invention permits an anti-inflammatory effect to be achieved.

In cases of inflammation, by using the device 201 it is in fact possible to activate the adenosinic receptors on the cell membrane via the generated biophysical stimulus.

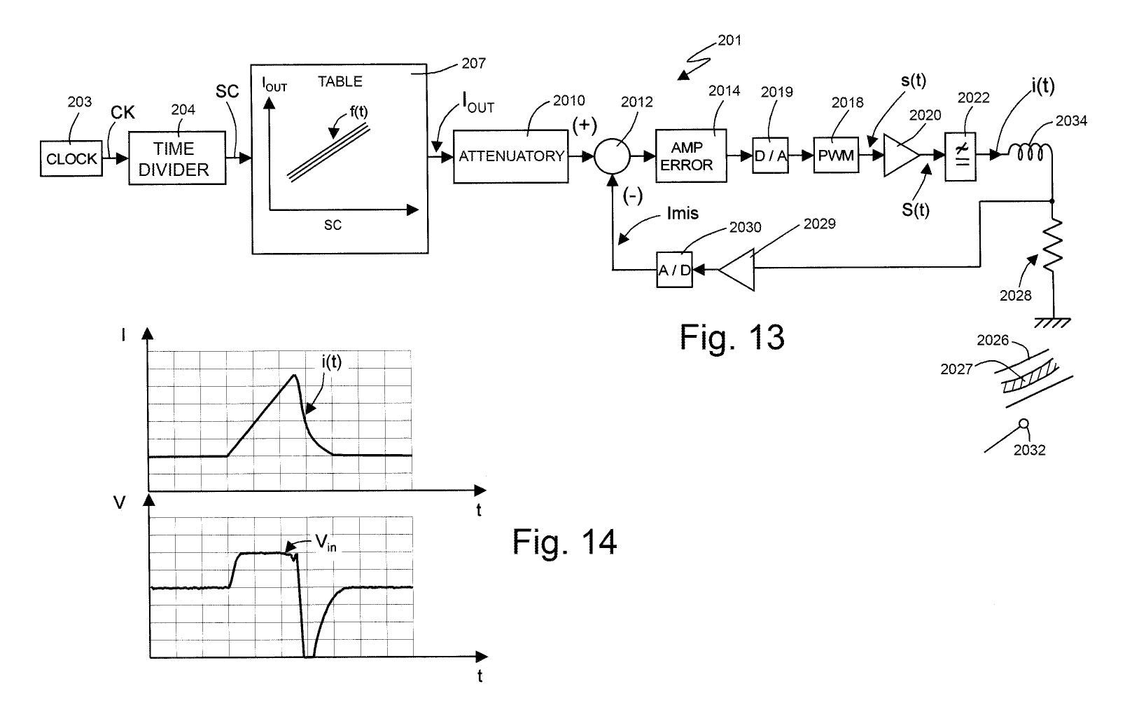

FIG. 15 shows in detail the transduction mechanism of the biophysical signal on the adenosinic receptors A2A of the cell membrane and the intracellular events activated by the bonding of adenosine with the associated receptor and generating the anti-inflammatory action.

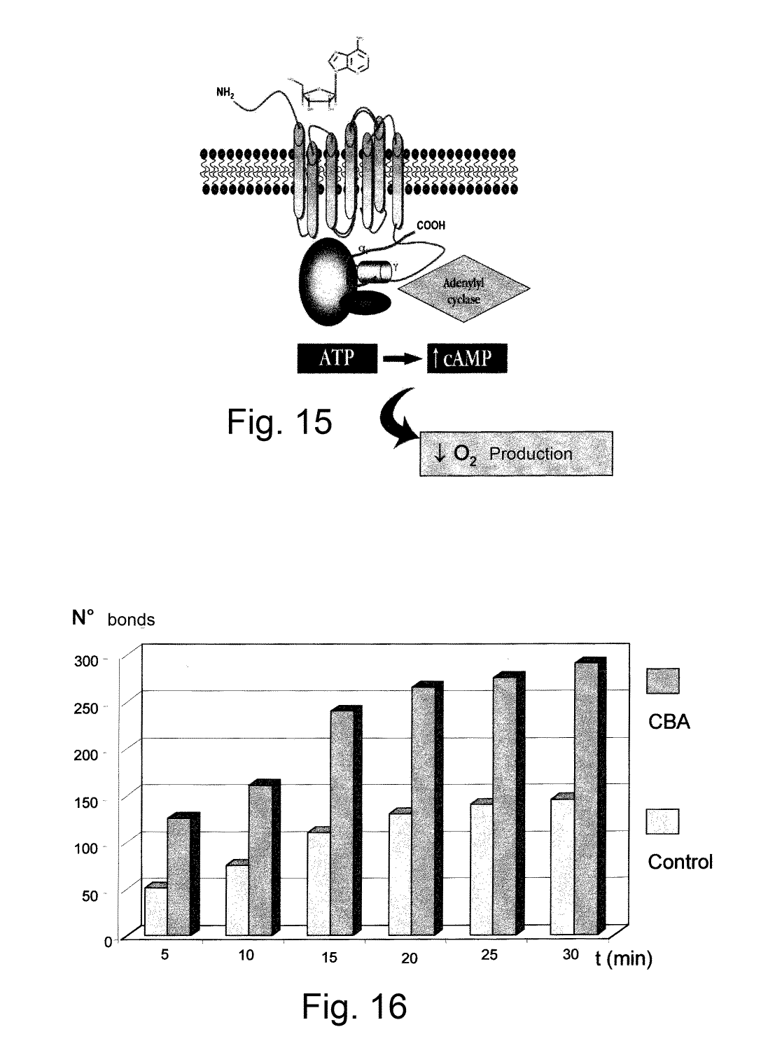

FIG. 16 shows instead a histogram representing the number of bonds formed between adenosine and the adenosinic receptor A2A on the membrane of human neutrophils in the presence of and in the absence of treatment with the device 201, as a function of time. As can be seen, the number of bonds formed and the consequent anti-inflammatory action is roughly doubled in the presence of the stimulation treatment provided via the device 201 realized according to the principles of this invention.

### In Vitro Effects

### Anabolic Effect on Cartilage

Anatomic Biophysical Chondroprotection exerts an anabolic action on cartilage in the presence of inflammatory cytokines (IL-1).

Explants of articular cartilage cultivated in the presence of inflammatory cytokines (IL-1) face an increase in catabolic activities, which accompanies degradation of the cartilaginous matrix and the decrease in synthesis of proteoglycans. If however, the explants are exposed to the electromagnetic field generated by the device 201 the catabolic effect of the inflammatory cytokines on the matrix is completely inhibited and the integrity of the cartilaginous matrix, as well as the proteoglycans synthesis capacity, is preserved.

FIG. 17 illustrates a histogram that shows the synthesis capacity of proteoglycans (S-PG) in explants of articular cartilage in the following conditions: control conditions, condition of exposure to the catabolic effect of the inflammatory cytokines and the condition of exposure to the catabolic effect of the inflammatory cytokines combined with stimulation via the device 201 in accordance with the invention. As can be noted, the synthesis capacity of proteoglycans is found to be heavily compromised due to the inflammatory cytokines, but returns to more-or-less normal values, equal to the control ones, in the presence of the Anatomic Biophysical Chondroprotection effect generated by the stimulation device 201.

### Example 4

### In Vivo Effects

### Inhibition of Articular Cartilage Degeneration

Anatomic Biophysical Chondroprotection inhibits the degenerative processes affecting articular cartilage that are observed with ageing. Utilizing the model of spontaneous osteoarthrosis in the guinea pig and quantifying the damage to the articular cartilage according to the Mankin classification, a strong chondroprotective effect linked with the stimulation supplied by the device 201 in accordance with the invention, was revealed.

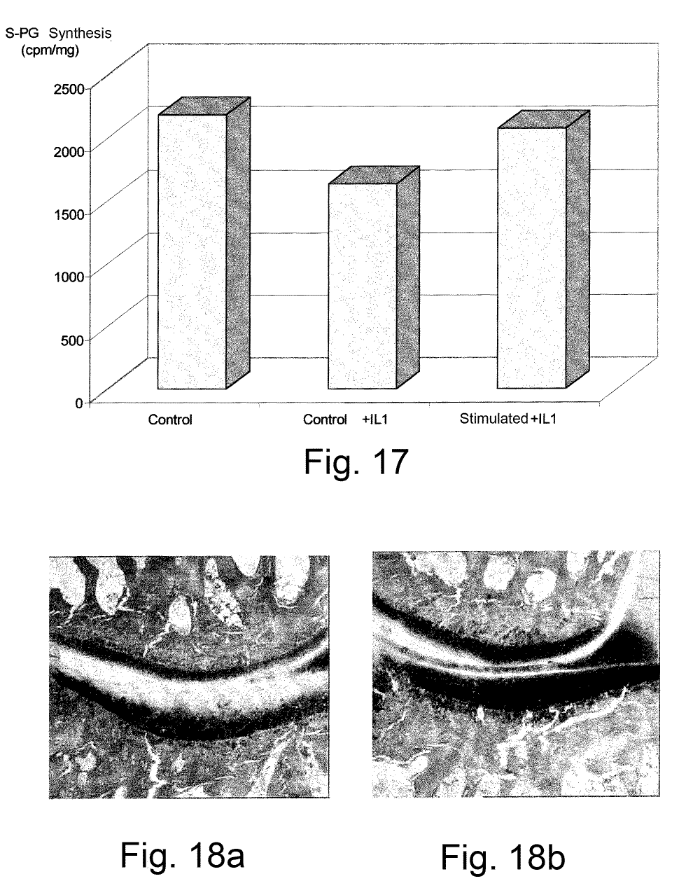

FIG. 18a shows evident signs of cartilage degeneration that are found in control animals due to ageing. FIG. 18b instead shows cartilage treated with the Anatomic Biophysical Chondroprotection therapy in which the absence of degeneration is evident. In particular, the thickness of the cartilage is maintained at normal levels, the colouring of the cartilaginous matrix appears intense and phenomena of fibrillation are not observed.

### Example 5

### In Vivo Effects

### Healing of Subchondral Bone Tissue

Anatomic Biophysical Chondroprotection exerts a healing action on subchondral bone tissue.

The healing of serious cartilage lesions can be carried out with various surgical options, the success on which depends in large measure to the characteristics of the subchondral bone tissue.

The action of the device 201 in accordance with this invention brings about rapid healing of subchondral bone tissue and prevents phenomena of bone reabsorption, creating optimal conditions for the viability of the overlaying articular cartilage. In addition, in the presence of a bone transplant, it favours the early anchorage of the graft itself, guarantees good integration of the transplanted bone tissue, prevents the formation of small bone cysts, and hence guarantees stability of the bone graft. It should be noted that in this regard, in the case of osteo-cartilaginous transplants, the early anchorage of subchondral bone is the necessary prerequisite for the viability and the preservation of the transplanted cartilage.

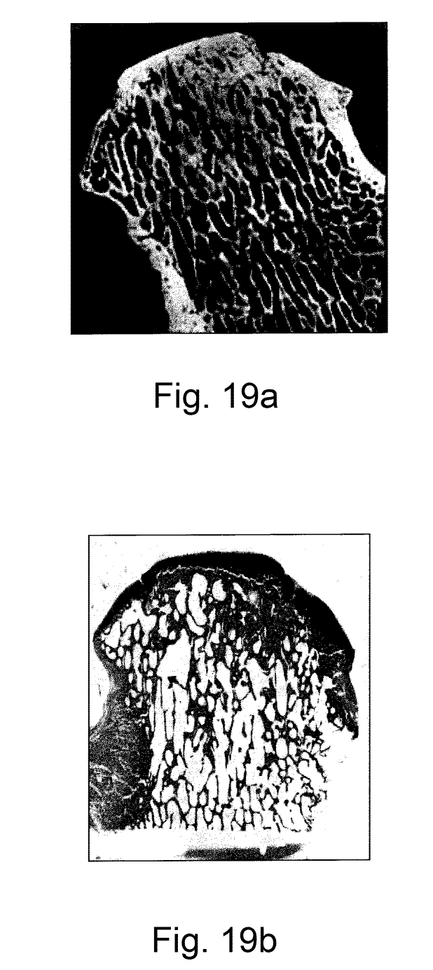

Two images are shown in FIGS. 19a and 19b regarding an osteo-cartilaginous graft on an animal model treated with the Anatomic Biophysical Chondroprotection therapy six months after the grafting operation. In particular, in the microradiographic image in FIG. 19a the complete integration of the subchondral bone may be noted, while in the histological image in FIG. 19b the viability of the transplanted cartilage can be observed, which exhibits an adequate thickness and intense colouring of the cartilaginous matrix.

From examination of the characteristics of the electromagnetic field stimulator device for Anatomic Biophysical Chondroprotection realized in accordance with this invention, the benefits that can be achieved with it are evident.

In particular, by using the above-described stimulator device, it is possible to programme the pilot current profile of the solenoid that generates the electromagnetic stimulation field point by point and it is also possible to create different current profiles by simply selecting different pilot maps, so as to take into account the various types of treatment and/or different solenoids used. In particular, the possibility of being able to realize an “ad hoc” current profile for each different solenoid used is advantageous.

Furthermore, this precise control of the pilot current permits an induced voltage to be generated that is as constant as possible and of adequate amplitude for the type of treatment.

Finally, via the implemented feedback system it is possible to react automatically to load changes, due to changes in impedance related to changes in temperature or to the tolerance of components for example, so as to ensure the stimulator device high operational stability and thus safeguard the therapeutic effectiveness of the stimulator device itself in all conditions.

1. The use of a device comprising:
means for generating a periodic signal/periodic signals u(t);
power amplifying means adapted to apply said signal u(t) to at least one solenoid for the generation of a pulsed electromagnetic field M(t) addressed towards a portion of human/animal tissue containing cartilage, to block or reduce the fibroblastic evolution of chondrocytes and mesenchymal cells.

2. The use according to claim 1, wherein it comprises setting means for the generation of an electromagnetic field having peak intensity in the range between 0.5 and 2 milliTesla.

3. The use according to claim 1, wherein the electromagnetic field has a power in the range between 1 and 2 milliTesla.

4. The use according to claim 1, wherein the electromagnetic field has a power of around 1.5 milliTesla.

5. The use according to claim 1, wherein said setting means (6, 4) contribute to generating a periodic signal u(t) having a frequency below 100 Hz.

6. The use according to claim 1, wherein said setting means contribute to generating a periodic signal u(t) having a frequency in the range between 2 and 75 Hz.

7. The use according to claim 1, wherein said setting means contribute to generating a periodic signal u(t) having a frequency in the range between 37 and 75 Hz.

8. The use according to claim 1, wherein said device is used in combination with a therapeutic agent.

9. The use according to claim 8, wherein said therapeutic agent is selected from the group consisting of growth factors, cytokines, base components of the extracellular matrix.

10. The use according to claim 1, wherein said portion of human/animal tissue containing cartilage contains a cartilaginous lesion subjected to a treatment by cellular therapy.

11. A method for blocking or reducing the fibroblastic evolution of chondrocytes and mesenchymal cells by means of electromagnetic field comprising the steps of:
generating a periodic signal/periodic signals u(t);
applying said signal u(t) to at least one solenoid for the generation of a pulsed electromagnetic field M(t) addressed towards a portion of human/animal tissue containing cartilage.

12. The method according to claim 11, wherein it comprises setting means for the generation of an electromagnetic field having a peak intensity in the range between 0.5 and 2 milliTesla.

13. The method according to claim 11, wherein the electromagnetic field has a power in the range between 1 and 2 milliTesla.

14. The method according to claim 11, wherein the electromagnetic field has a power of around 1.5 milliTesla.

15. The method according to claim 11, wherein said setting means contribute to generating a periodic signal u(t) having a frequency below 100 Hz.

16. The method according to claim 11, wherein said setting means contribute to generating a periodic signal u(t) having a frequency in the range between 2 and 75 Hz.

17. The method according to claim 11, wherein said setting means contribute to generating a periodic signal u(t) having a frequency in the range between 37 and 75 Hz.

18. The method according to claim 11, wherein said device is used in combination with a therapeutic agent.

19. The method according to claim 18, wherein said therapeutic agent is selected from the group consisting of growth factors, cytokines, base components of the extracellular matrix.

20. Method according to claim 11, wherein said portion of human/animal tissue containing cartilage contains a cartilaginous lesion subjected to a treatment by cellular therapy.
